(19) United States
(12) Patent Application Publication
(10) Pub. No.: US 2025/0366825 A1
(43) Pub. Date: Dec. 4, 2025

(54) MEDICAL DEVICES WITH ENHANCED ECHOGENICITY

(71) Applicant: BOSTON SCIENTIFIC SCIMED, INC., Maple Grove, MN (US)

(72) Inventors: CRISTINA ROMANY, Orlando, FL (US); DANIELLE FRANKSON, Dayton, MN (US); GENE THOMAS STORBECK, Millis, MA (US)

(73) Assignee: BOSTON SCIENTIFIC SCIMED, INC., Maple Grove, MN (US)

(21) Appl. No.: 19/224,038

(22) Filed: May 30, 2025

Related U.S. Application Data
(60) Provisional application No. 63/654,483, filed on May 31, 2024.

(51) Int. Cl. A61B 8/00; A61F 2/958; A61M 25/00

(52) U.S. Cl. CPC A61B 8/445; A61F 2/958; A61M 25/0045

(57) ABSTRACT

Medical devices including medical devices with enhanced echogenicity are disclosed. An example medical device may include a polymeric catheter shaft having a distal end region. The distal end region may include a plurality of hyperechoic particles disposed therein.

### CROSS-REFERENCE TO RELATED APPLICATIONS

The application claims the benefit of U.S. Provisional Patent Application Ser. No. 63/654,483, filed on May 31, 2024, the disclosure of which is incorporated herein by reference.

### TECHNICAL FIELD

The present disclosure pertains to medical devices, and methods for manufacturing medical devices. More particularly, the present disclosure pertains to medical devices with enhanced echogenicity.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device with enhanced echogenicity is disclosed. The medical device comprises: a polymeric catheter shaft having a distal end region; and wherein the distal end region includes a plurality of hyperechoic particles disposed therein.

Alternatively or additionally to any of the embodiments above, the plurality of hyperechoic particles includes microspheres.

Alternatively or additionally to any of the embodiments above, the plurality of hyperechoic particles includes hollow microspheres.

Alternatively or additionally to any of the embodiments above, the plurality of hyperechoic particles includes hollow glass microspheres.

A medical device with enhanced visualization properties is disclosed. The medical device comprises: a medical device body configured to be disposed within a body lumen, the medical device body including a hyperechoic region comprising a polymer and a scattering member configured to scatter ultrasonic energy in order to enhance ultrasonic visualization.

Alternatively or additionally to any of the embodiments above, the medical device body includes an access canula.

Alternatively or additionally to any of the embodiments above, the medical device body includes a catheter shaft.

Alternatively or additionally to any of the embodiments above, the medical device body includes a section of a balloon catheter.

Alternatively or additionally to any of the embodiments above, the medical device body includes a section of a retrieval basket or a retrieval snare.

Alternatively or additionally to any of the embodiments above, the medical device body includes a stent or a section of a stent delivery system.

Alternatively or additionally to any of the embodiments above, the medical device body includes a catheter shaft comprising an inner layer, an outer layer, and an undulating layer disposed between the inner layer and the outer layer.

Alternatively or additionally to any of the embodiments above, the undulating layer includes one or more undulations.

Alternatively or additionally to any of the embodiments above, the undulating layer defines one or more air pockets along the catheter shaft.

Alternatively or additionally to any of the embodiments above, the medical device body includes a plurality of hyperechoic particles.

Alternatively or additionally to any of the embodiments above, the plurality of hyperechoic particles includes microspheres.

Alternatively or additionally to any of the embodiments above, the plurality of hyperechoic particles includes hollow microspheres.

Alternatively or additionally to any of the embodiments above, the plurality of hyperechoic particles includes hollow glass microspheres.

A medical device is disclosed. The medical device comprises: a catheter shaft including an inner layer, an outer layer, and an undulating member disposed between the inner layer and the outer layer; and wherein the undulating member defines a plurality of air pockets within the catheter shaft that are configured to scatter ultrasonic energy in order to enhance ultrasonic visualization of the catheter shaft.

Alternatively or additionally to any of the embodiments above, the undulating member includes a plurality of axially-extending undulations.

Alternatively or additionally to any of the embodiments above, the undulating member includes a plurality of radially-extending undulations.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic depiction of an access device being used to access a target region.

FIG. 2 schematically depicts an example ultrasound display.

FIG. 3 is a cross-sectional view of an example medical device body.

FIG. 4 schematically depicts a region of an example medical device body.

FIG. 5 schematically depicts an example ultrasound display.

FIG. 6 is a cross-sectional view of a portion of an example medical device body.

FIG. 7 is a cross-sectional view of a portion of an example medical device body.

FIG. 8 is a side view of a portion of an example medical device body.

FIG. 9 is a perspective view of a portion of an example medical device body.

FIG. 10 is a perspective view of a portion of an example medical device body.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “of” is generally employed in its sense including “and/of” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

A number of medical interventions utilize ultrasound to help guide and/or visualize a medical device and/or target anatomy. For example, endoscopic ultrasound (EUS) procedures may be performed with a specialized scope that uses high frequency soundwaves to visualize nearby structures. The relative density and geometry of items in field view play a role in EUS “visibility” such that features like air pockets appear brightly lit on the feedback screen.

Some EUS include treating patients where endoscopic retrograde cholangiopancreatography (ERCP) for biliary drainage fails. EUS can be used to recover these failed ERCP procedures either through a recover rendezvous procedure or direct biliary drainage. Such procedures may start with an EUS access procedure to gain guidewire access into the common bile, intrahepatic, or pancreatic ducts. During these procedures, a device may be passed through the working channel of a specialized scope. The device may be used to puncture and cannulate the target anatomy in preparation for a guidewire to gain access (through and anchored by the access cannula). In some instances, puncture and cannulation are performed together by a sharp and access cannula, before the sharp is retracted to allow for passage of a guidewire, through the cannula and into the patient target anatomy.

Metallic sharps, which may include echogenic features, may have a tendency to scatter ultrasonic energy, thereby allowing for suitable visualization. Access cannulas may be made from polymeric materials, which may have a lower tendency to scatter ultrasonic energy and, thus, may harder to visualize with ultrasound. Consequently, fluoroscopic visualization may be used to visualize the access cannula and determine the position of the sharp and/or access cannula relative to one another (e.g., including sharp offset) and/or the anatomy. Disclosed herein are medical devices that are designed to have enhanced echogenicity. This may include medical devices such as access cannulas, catheters (including balloon catheters), snare and/or basket devices, delivery systems, stents, and/or the like.

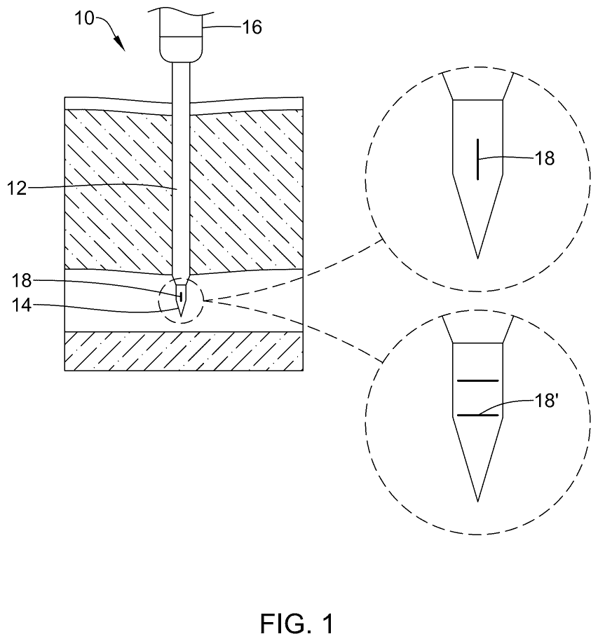

FIG. 1 schematically depicts a system 10 for a medical intervention. In this example, an EUS procedure is depicted where a catheter or access cannula 12 is used to access a target location. A sharp or puncture member 14 may be disposed within the access cannula 12. In some instances, a guiding device or scope 16 may be used to guide the access cannula 12. The guiding device or scope 16 may be component of the access cannula 12. Alternatively, the guiding device or scope 16 may be a separate device, for example that can be used with the access cannula 12.

In some instances, the sharp 14 may include one or more echogenic features. For example, the sharp 14 may include laser cuts and/or markings 18, 18′. Different arrangements of the cuts/markings 18, 18′ are schematically depicted in FIG. 1. For example, in some instances a singular axial (e.g., vertical) cut 18 may be formed in the sharp 14. Alternatively, multiple cuts such as transverse cuts 18′ may be formed in the sharp 14. It can be appreciated that a variety of a cuts and/or markers can be utilized for the sharp 14, for example to increase the echogenicity of the sharp 14.

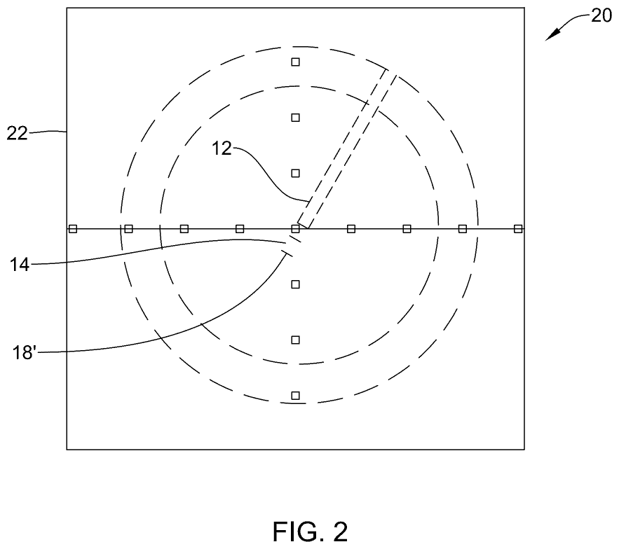

FIG. 2 schematically depicts an example ultrasound display system 20. The system 20 may include a display 22. As shown on the display, the distal the sharp 14 may be visible. For example, the markings 18′ may be visible on the display 22. The access cannula 12 may also visible, but to a lesser extent as represented in FIG. 2 by dashed/phantom lines. To further visualize the access cannula 12, fluoroscopic visualization processes may be utilized.

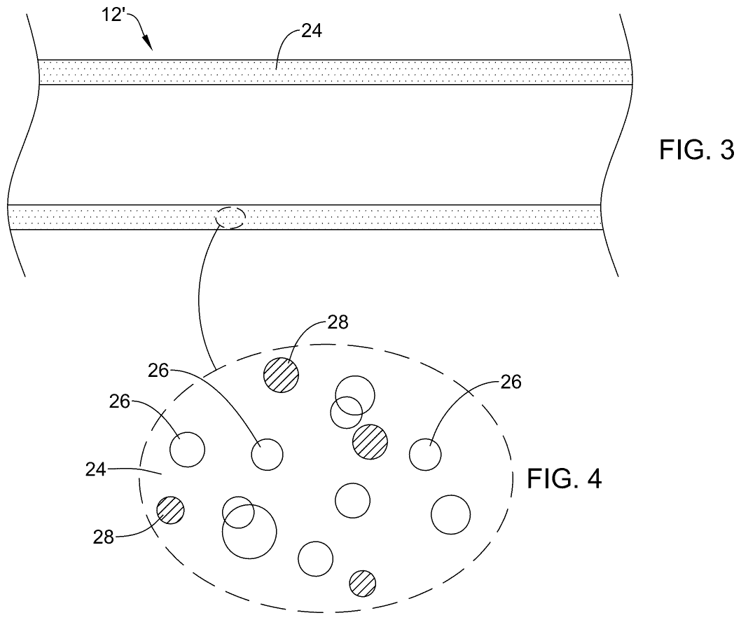

As indicated herein, it may be desirable to enhance echogenicity of various medical devices so that such devices may be efficiently visualized using ultrasound. For example, FIG. 3 illustrates another example medical device or shaft 12′, which may be similar in form and function to other devices disclosed herein. In this example, the shaft 12′ may take the form of a tube. The tube 12′ may be a catheter, access cannula, and/or another similar medical device.

The tube 12′ may include a polymeric substrate or resin 24 having plurality of echogenic particles 26 therein as shown in FIG. 4. Such particles 26 may take the form of microspheres, nanospheres, hollow microspheres, hollow nanospheres, glass microspheres, glass nanospheres, hollow glass microspheres, hollow glass nanospheres, air pockets, combinations thereof, glass, polymeric particles, ceramic materials, metallic particles, salt, blowing agents, a microlumen (e.g., a relatively small passageway or lumen formed into the tube wall), a nanotube (e.g., a carbon nanotube), a composite material (e.g., carbon fiber), combinations thereof, and/or the like. The echogenic particles 26 may have a suitable size such as about 1-500 microns or about 5-150 microns. The echogenic particles 26 may be similar or uniform in size. Alternatively, the echogenic particles 26 may differ in size. In instances where hollow spheres are utilized, the wall thickness of the spheres may be tuned to provide the desired echogenicity.

In one example, glass, polymer, ceramic, metal, or the like hollow microspheres may be used. Using hollow spheres may help to maintain a favorable (e.g. low) weight. Such materials/spheres may be utilized when forming tube 12′ via an extrusion, molding, and/or the like. In addition or in the alternative, such materials may be used with dip coatings. Either way, the microspheres (e.g., hollow microspheres) may increase the echoic behavior under ultrasound viewing.

In another example, salt such as sodium chloride may be disposed within the resin 24 (e.g., via subfusion), for example during an extrusion process, in order to produce intentional air pockets. Such air pockets may increase the echoic behavior of the shaft 12′.

In some instances, an additional lumen may be incorporated into the tube 12′, for example in an extrusion process, that can form/include air pockets. Such additional lumens may be sufficiently small to fit into the tube wall and, generally, would not be used to pass another device therethrough but rather would be used for air pockets to increase echoic behavior. Similarly, nanotubes such as carbon nanotubes may be incorporated into molded or dipped parts. Such nanotubes may be randomly oriented to increase reflectance properties and/or increase echoic behavior. In some cases, woven or multilayer structures (e.g., which may include carbon fiber) may incorporated into the tube 12′. Such woven structures may have localized density variations and/or structural geometries, which may enhance echogenicity.

As indicated herein, forming the tube 12′ may include a suitable process. For example, the resin 24 and echogenic particles 26 may be combined/mixed. The ratio or relative amount/number of echogenic particles 26 to resin material may be varied or tuned in order to provide the desired echogenicity. The mixed resin 24 and echogenic particles 26 may be formed into the tube 12′ by an extrusion process, molding process, casting process, and/or other suitable processes. The resin 24 may include a suitable material or materials such as those disclosed herein. For example, the resin 24 may include polyether ether ketone (e.g., VICTREX 650g), nylon (e.g., GRIVORY 21), polyethylene, high-density polyethylene, fluoropolymers (e.g., polytetrafluoroethylene), polymethyl methacrylate, polyether sulfone, polyether block amide, polyether-ester, combinations thereof, and/or the like. In some instances, the resin 24 may also have other materials or particles 28 therein. For example, radiopaque particles 28 may be disposed within the resin 24. In some of these and in other instances, radiopaque fillers can be added/compounded with the resin 24, for example, to increase the fluoroscopic visualization characteristics.

In some instances, the echogenic particles 26 may be disposed along an entire length of the shaft 12′. Alternatively, the echogenic particles 26 may be disposed along one or more discrete lengths or regions of the shaft 12′. In examples where the echogenic particles 26 are disposed along one or more discrete lengths or regions of the shaft 12′, echo transparent regions (e.g., regions transparent to ultrasound) may be disposed between the echoic region (e.g., including the echogenic particles 26). This may allow regions of the shaft 12′ to be arranged and/or used akin to an echogenic ruler for taking measurements within a patient.

Rather than being formed as a tube or shaft 12′, the echogenic particles may be incorporated into relatively short sleeves or bands that can be applied to a medical device in order to enhance echogenicity. For example, echogenic bands, which may be similar in form to typical radiopaque marker bands, may be incorporated into a variety of medical devices in order to enhance echogenicity.

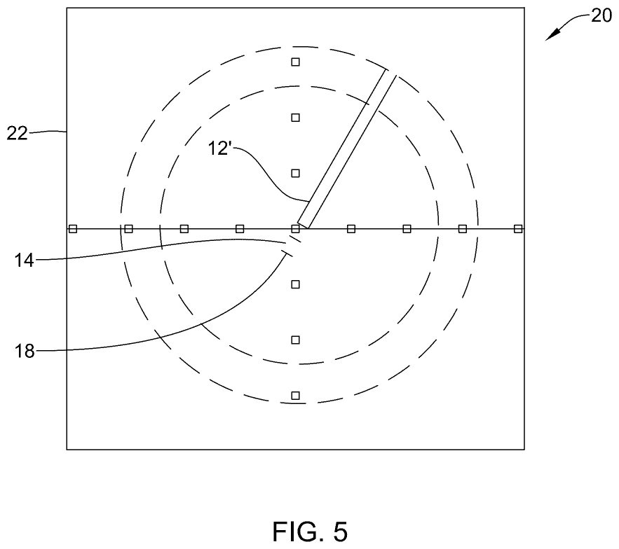

The echogenic particles 26 may be configured to enhance echogenicity, for example, by encouraging the scatter of ultrasound energy in a manner similar to air pockets (e.g., air pockets within dimpled features), laser cuts, markings, etc. For example, as schematically shown in FIG. 5, the shaft 12′ may have desirable echogenicity as represented by solid lines.

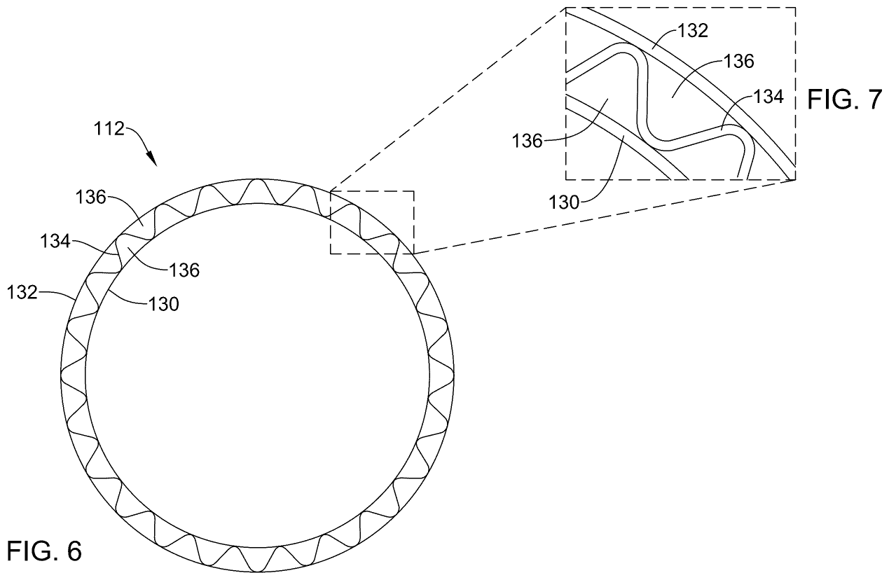

FIGS. 6-7 illustrate a portion of another example medical device 112, which may be similar in form and function to other devices disclosed herein. In this example, the medical device 112 may take the form of a tube that includes an inner layer 130, an outer layer 132, and a textured or undulating member 134 disposed between the inner layer and the outer layer 132. The undulating member 134 may include a plurality of axially-extending and/or radially-extending undulations or waves. The shape/arrangement of the undulating member 134 within the medical device 112 may form or define one or more air pockets 136 within the medical device 112 (e.g., within the wall of the medical device 112). The air pockets 136 may enhance the echogenicity of the medical device 112.

In some of these and in other instances, the undulating member 134 may include a porous material, for example disposed between the inner and outer layers 130, 132. The porous material/layer, which may or may not include undulations, may include a suitable material such as expanded polytetrafluoroethylene. In some of these and in other instances, the inner and outer layers 130, 132 may include materials such as those disclosed herein such as polyetheretherketone.

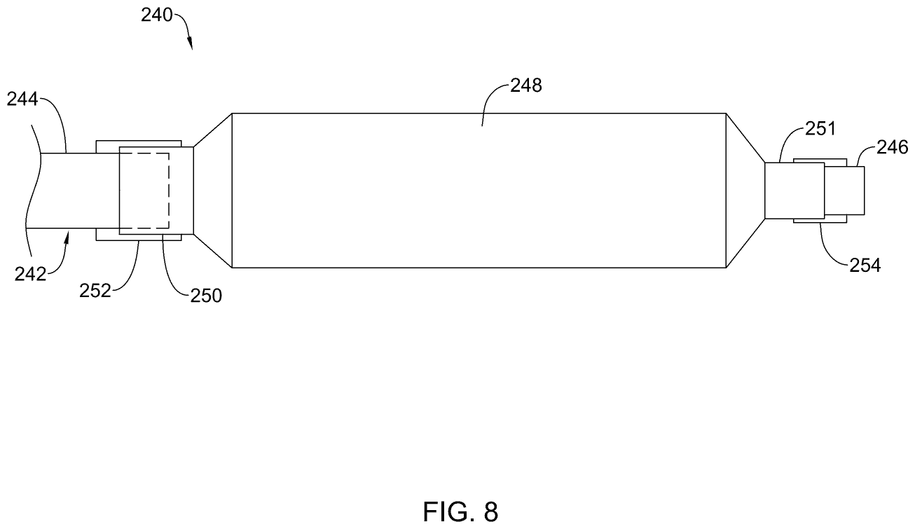

As indicated herein, it may be desirable to incorporate echoic properties into a wide variety of different medical devices. A few example applications are disclosed in FIGS. 8-10. Other applications are contemplated. For example, FIG. 8 illustrates a portion of another example medical device 240, which may be similar in form and function to other devices disclosed herein. In this example, the medical device 240 may be a balloon catheter. The balloon catheter may include a catheter shaft 242 including an outer shaft 244 and an inner shaft 246. A balloon 248 may be coupled to the catheter shaft 242. One or more echogenic members may be coupled to the medical device 240. For example, an echogenic member 252 may be coupled to the balloon 248 and/or the catheter shaft 242. The echogenic member 252 may take the form of a sleeve or covering disposed along discrete portions of the balloon catheter. For example, the echogenic member 252 may be disposed along the proximal waist 250 of the balloon 248 and/or the outer shaft 244. In some of these and in other instances, an echogenic member 254 may be coupled to the balloon 248 and/or the catheter shaft 242. For example, the echogenic member 254 may be disposed along the distal waist 251 of the balloon 248 and/or the inner shaft 246. The echogenic members 252 may be structural similar to other echogenic structures disclosed herein. For example, the echogenic member 252 may include echogenic particles.

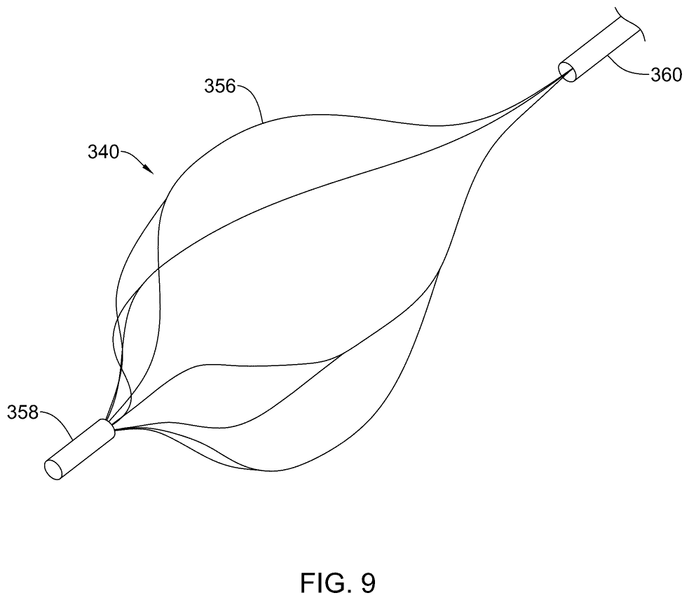

FIG. 9 illustrates a portion of another example medical device 340, which may be similar in form and function to other devices disclosed herein. In this example, the medical device 340 may be a basket or snare device. The basket device may include a basket 356. One or more echogenic members may be coupled to the medical device 340. For example, an echogenic member 358 may be disposed at the distal end of the basket 356. In some of these and in other instances, an echogenic member 360 may be disposed at the proximal end of the basket 356. The echogenic members 352 may be structural similar to other echogenic structures disclosed herein. For example, the echogenic member 352 may include echogenic particles.

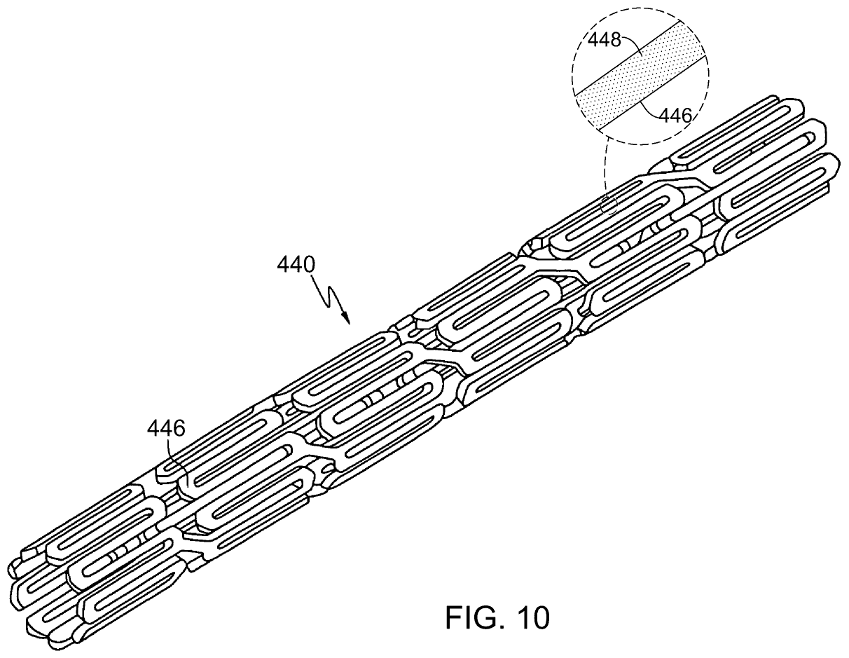

FIG. 10 illustrates a portion of another example medical device 440, which may be similar in form and function to other devices disclosed herein. In this example, the medical device 440 may be a stent. The stent may include one or more struts 446. One or more echogenic members may be coupled to the medical device 440. For example, an echogenic member 448 may be disposed along the struts 446. In some instances, the struts 446 may include echogenic particles therein. In some of these and in other instances, the struts 446 may include a covering or coating (e.g., dip coating) that is configured to enhance the echogenicity of the medical device 440.

The materials that can be used for the various components of the system 10 (and/or other systems disclosed herein) may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively high contrast image on a fluoroscopy screen or another imaging technique during a medical procedure. This relatively high contrast image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device with enhanced echogenicity, comprising:
a polymeric catheter shaft having a distal end region; and
wherein the distal end region includes a plurality of hyperechoic particles disposed therein.

2. The medical device of claim 1, wherein the plurality of hyperechoic particles includes microspheres.

3. The medical device of claim 1, wherein the plurality of hyperechoic particles includes hollow microspheres.

4. The medical device of claim 1, wherein the plurality of hyperechoic particles includes hollow glass microspheres.

5. A medical device with enhanced visualization properties, the medical device comprising:
a medical device body configured to be disposed within a body lumen, the medical device body including a hyperechoic region comprising a polymer and a scattering member configured to scatter ultrasonic energy in order to enhance ultrasonic visualization.

6. The medical device of claim 5, wherein the medical device body includes an access canula.

7. The medical device of claim 5, wherein the medical device body includes a catheter shaft.

8. The medical device of claim 5, wherein the medical device body includes a section of a balloon catheter.

9. The medical device of claim 5, wherein the medical device body includes a section of a retrieval basket or a retrieval snare.

10. The medical device of claim 5, wherein the medical device body includes a stent or a section of a stent delivery system.

11. The medical device of claim 5, wherein the medical device body includes a catheter shaft comprising an inner layer, an outer layer, and an undulating layer disposed between the inner layer and the outer layer.

12. The medical device of claim 11, wherein the undulating layer includes one or more undulations.

13. The medical device of claim 11, wherein the undulating layer defines one or more air pockets along the catheter shaft.

14. The medical device of claim 5, wherein the medical device body includes a plurality of hyperechoic particles.

15. The medical device of claim 14, wherein the plurality of hyperechoic particles includes microspheres.

16. The medical device of claim 14, wherein the plurality of hyperechoic particles includes hollow microspheres.

17. The medical device of claim 14, wherein the plurality of hyperechoic particles includes hollow glass microspheres.

18. A medical device, comprising:
a catheter shaft including an inner layer, an outer layer, and an undulating member disposed between the inner layer and the outer layer; and
wherein the undulating member defines a plurality of air pockets within the catheter shaft that are configured to scatter ultrasonic energy in order to enhance ultrasonic visualization of the catheter shaft.

19. The medical device of claim 18, wherein the undulating member includes a plurality of axially-extending undulations.

20. The medical device of claim 18, wherein the undulating member includes a plurality of radially-extending undulations.
